(19) United States
(12) Patent Application Publication
(10) Pub. No.: US 2006/0223882 A1
(43) Pub. Date: Oct. 5, 2006

(54) Amorphous simvastatin

(76) Inventors: Venkataraman Sundaram, Hyderabad (IN); Manoj Ramesh Kharkar, Hyderabad (IN); Sesha Reddy Yarraguntla, Hyderabad (IN); Srinivasulu Gudipati, Hyderabad (IN); Venkata Naga Brahmeswara Rao Mandava, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/386,962

(22) Filed: Mar. 22, 2006

Related U.S. Application Data
(60) Provisional application No. 60/678,013, filed on May 5, 2005.

(30) Foreign Application Priority Data
Mar. 23, 2005 (IN) 306/CHE/2005

(51) Int. Cl. C07D 309/30; A61K 31/366

(52) U.S. Cl. 514/460; 549/292

(57) ABSTRACT

Amorphous simvastatin is prepared by rapidly removing solvent from a solution comprising simvastatin. The solution can also comprise a pharmaceutically acceptable carrier, to form a dispersion of amorphous simvastatin in the carrier.

### CROSS-REFERENCE TO RELATED APPLICATION

The present application is a nonprovisional filing of copending U.S. Provisional Patent Application No. 60/678,013 filed May 5, 2005, the entire content of which is incorporated herein by this reference.

### INTRODUCTION TO THE INVENTION

The present invention relates to an amorphous form of simvastatin and a process for the preparation thereof.

Simvastatin is chemically known as 2,2-dimethylbutanoic acid (1S,3R,7S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester, or butanoic acid, 2,2-dimethyl-1,2,3,7,8,8a-hexahydro-3,7-dimethyl-8-[2-(tetrahydro-4-hydroxy-6-oxo-2H-pyran-2-yl)-ethyl]-1-naphthalenyl ester, [1S-[1α,3α,7β,8β(2S*,4S*),-8aβ]] and is represented by the structural Formula I.

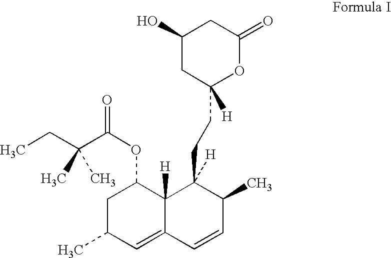

Simvastatin is a lipid-lowering agent that is derived synthetically from a fermentation product of Aspergillus terreus. After oral ingestion, simvastatin, which is an inactive lactone, is hydrolyzed to the corresponding β-hydroxy acid form. This is an inhibitor of 3-hydroxy-3-methylglutaryl-coenzyme A (HMG-CoA) reductase. This enzyme catalyzes the conversion of HMG-CoA to mevalonate, which is an early and rate-limiting step in the biosynthesis of cholesterol. Pharmaceutical products containing simvastatin as the active ingredient are commercially available in the market as ZOCOR™ in the form of solid oral formulations containing either 5 mg, 10 mg, 20 mg, 40 mg or 80 mg as tablets.

U.S. Pat. No. 4,444,784 discloses simvastatin and its pharmaceutical compositions. It also describes the process for synthesis of simvastatin, which involves deacylation of lovastatin followed by a subsequent acylation with the 2,2-dimethylbutyryl moiety.

PCT Application Publication No. WO 00/53566 discloses a crystalline form of calcium salt of dihydroxy open-acid of simvastatin and a process for the preparation thereof, particularly hydrated calcium salts characterized by corresponding x-ray powder diffraction (XRPD), thermogravimetry (TGA), differential scanning calorimetry (DSC) and solid-state C¹³ NMR spectroscopy data.

PCT Application Publication No. WO 02/20457 discloses five crystalline polymorphic forms of simvastatin calcium salt, forms I, II, III, IV and V, including both hydrated and anhydrous forms, their preparation and characterization by XRPD, TGA, DSC and solid-state C¹³ NMR spectroscopy. Each of these crystalline forms, having different degrees of hydration, is prepared using different drying methods.

PCT Application Publication No. WO 04/089868 A1 discloses the anhydrous and hydrated amorphous simvastatin calcium and a process for the preparation thereof.

PCT Application Publication No. WO 04/069819 A1 also discloses crystalline simvastatin and the preparation thereof.

There remains a need for new forms of simvastatin, in particular forms suitable for preparing rapid-onset compositions, exists. Rapid-onset drug-delivery systems can provide significant benefits over conventional dosage forms. Generally, rapid-onset preparations provide a short period to therapeutic or prophylactic response compared to conventional immediate-release or sustained-release dosage forms.

The amorphous forms of a number of drugs exhibit different dissolution characteristics, generally enhanced dissolution, resulting frequently in an enhanced bioavailability profile compared to crystalline forms of the same drugs. Generally, a higher bioavailability would result in a lower dose being administered to the patient thus further resulting in a better economic value to the customer. Also, a lower dose administered could possibly result in reduced toxicities, providing a further benefit to the patient.

However, simvastatin presents certain challenges for formulation as a rapid-onset dosage form, particularly as a rapid-onset oral dosage form. For example, simvastatin has a very low solubility in aqueous media (practically insoluble) and therefore is not readily dissolved and dispersed for rapid absorption in the gastrointestinal tract when administered orally, for example in tablet or capsule form.

The bioavailability of an orally administered drug, as measured by its entry into systemic circulation in the bloodstream, depends on at least two fundamental processes: drug dissolution in gastrointestinal fluids (in vivo drug release) and subsequent absorption of the dissolved drug. Several factors influence dissolution of a drug from its carrier, including surface area of the drug presented to the dissolution solvent medium, solubility of the drug substance in the solvent medium, and driving forces of the saturation concentration of dissolved materials in the solvent medium. For simvastatin, where its principle site of action is the liver, a more rapid and complete absorption would definitely help therapy.

Crystalline solids, due to their highly organized, lattice-like structures, typically require a significant amount of energy for dissolution. The energy required for a drug molecule to escape from a crystal, for example, is greater than is required for the same drug molecule to escape from a non-crystalline, amorphous form. Importantly, however, crystalline drug forms, which have been transformed into amorphous forms, tend to revert to a steady state of low energy, namely the crystalline form, over time and thus may not have an adequate shelf life.

Therefore, if an amorphous form of simvastatin could be prepared, and in particular if a storage-stable amorphous form of simvastatin could be developed exhibiting enhanced bioavailability, for example through rapid dissolution of the drug, a significant advance would be realized in the treatment of hypercholesterolemia.

### SUMMARY OF THE INVENTION

The present invention relates to amorphous simvastatin or an amorphous combination of simvastatin and a pharmaceutically acceptable carrier. The amorphous simvastatin of the present invention is characterized by the XRPD pattern as shown in FIG. 1.

The present invention further relates to a process for the preparation of amorphous simvastatin or an amorphous combination of simvastatin and pharmaceutically acceptable carriers. Such amorphous simvastatin can be obtained by drying of a solution of simvastatin in an organic solvent alone or simvastatin in combination with suitable pharmaceutical carriers in an organic solvent.

### BRIEF DESCRIPTION OF THE DRAWING

FIG. 1 is an X-ray powder diffraction (“XRPD”) pattern for the amorphous form of simvastatin prepared in Example 1.

### DETAILED DESCRIPTION

Unless defined otherwise, all technical and scientific terms used herein have the same meaning as commonly understood by one of ordinary skill in the art to which this invention belongs. The phrase “amorphous simvastatin” is intended to include any amorphous form of simvastatin including but not limited to amorphous simvastatin, an amorphous solid dispersion of simvastatin, and amorphous combinations of simvastatin with pharmaceutically acceptable carriers or crystallization inhibitors.

It has been found that obtaining an amorphous solid form of simvastatin is not a simple matter. Amorphous simvastatin may be obtained with or without using suitable pharmaceutical carriers. When prepared without the presence of pharmaceutical carriers, the simvastatin may be dissolved in an organic solvent and the solution thus obtained may be dried by any known conventional means of drying. A mixture of organic solvents may be used. It is generally preferred that a rapid drying be utilized to provide the desired amorphous form substantially free from moisture and organic solvent.

Amorphous simvastatin may also be prepared in the presence of pharmaceutically acceptable carriers or crystallization inhibitors. Without being bound by theory, it is expected that the drug is dispersed within the polymer at a molecular level resulting in an amorphous material. Thus, for such a material the energy required for breaking down the crystal structure to bring the drug into solution is reduced, thereby resulting in enhanced solubility, more rapid dissolution or both. Further, such materials also act as crystallization inhibitors to prevent the conversion of the novel amorphous form of simvastatin into a crystalline form thus resulting in enhanced stability of the compound at conventional storage temperatures.

Such amorphous simvastatin along with a carrier may be prepared by providing a solution of simvastatin in a organic solvent, dissolving the pharmaceutically acceptable carrier in a second organic solvent, mixing the two solutions, and subjecting this solution to solvent removal by any conventional means of drying. The organic solvent used for dissolving the simvastatin and the pharmaceutically acceptable carrier may be the same or different ones may be used. In both the above-mentioned cases, in some cases it may be required to raise the temperature to enhance the dissolution of the simvastatin and the pharmaceutically acceptable carrier. Any temperature is acceptable as long as this does not cause degradation of the product.

Of course, the rates of drying, the concentrations of the respective solutions, and the weight ratios of the simvastatin to the pharmaceutically acceptable carrier to achieve a stable amorphous product can be defined by a person skilled in the art and are all included herein without limitation. In general, techniques that provide rapid removal of solvent from a simvastatin solution will prepare amorphous simvastatin.

The pharmaceutically acceptable carriers that can be used for the preparation of amorphous simvastatin include but are not limited to pharmaceutical hydrophilic carriers such as polyvinylpyrrolidone (homopolymers or copolymers of N-vinylpyrrolidone), gums, cellulose derivatives (including hydroxypropyl methylcellulose, hydroxypropyl cellulose and others), cyclodextrins, gelatins, hypromellose phthalate, sugars, polyhydric alcohols, polyethylene glycol, polyethylene oxides, polyoxyethylene derivatives, polyvinyl alcohol, propylene glycol derivatives and the like. The use of mixtures of more than one of the pharmaceutical carriers to provide desired release profiles or for the enhancement of stability is within the scope of this invention. Also, all viscosity grades, molecular weights, commercially available products, their copolymers, mixtures are all within the scope of this invention without limitation.

Organic solvents useful for preparing amorphous simvastatin are generally good solvents for the simvastatin and also for the pharmaceutically acceptable carrier, if it is to be used. Examples of such solvents include but are not limited to any solvent or mixture of solvents in which simvastatin is soluble, such as: alcoholic solvents like methanol, ethanol and the like; halogenated solvents such as dichloromethane, chloroform, ethylene dichloride and the like; ketonic solvents such as acetone, ethyl, methyl ketone and the like; esters such as ethyl acetate, n-butyl acetate, t-butyl acetate and the like; ether solvents such as diethyleter, diisopropyl ether, methyl tertiary butyl ether, tetrahydrofuran, and the like; hydrocarbon solvents like toluene or xylene; and the like. The same or different solvent(s) may be used for the simvastatin and for the pharmaceutically acceptable carrier. Any solvent is acceptable as long as it provides a clear solution of the simvastatin alone or in combination with the pharmaceutically acceptable carrier, when present, at the temperature of operation.

A particularly useful solvent for dissolution of simvastatin with or without a suitable pharmaceutical carrier is methanol. The dissolution temperature can range from 20 to 120° C.

Any form of simvastatin known in the art is acceptable as a starting material for the processes of this invention. This includes without limitation any of the crystalline forms described in the prior art or any possible solvates, hydrates or crystalline forms possible for simvastatin. Salts of simvastatin may be used.

The resultant solution can be dried by using any conventional methods of drying including spray drying, rotational evaporation (such as using a Buchi Rotavapor), agitated thin film drying-vertical (ATFD-V), spin-flash drying, fluid-bed drying, lyophilization or other techniques known in art.

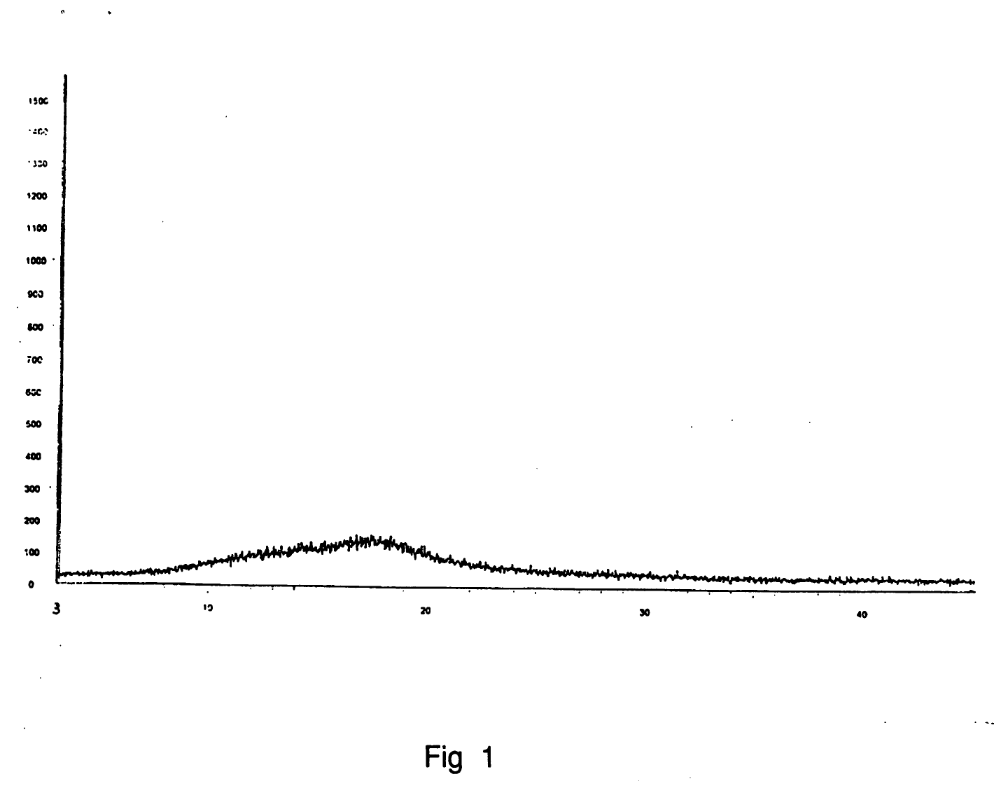

The XRPD pattern of the amorphous simvastatin was measured on a Bruker Axe, DS Advance Powder X-ray Powder Diffractometer using a Copper K alpha-1 radiation source. Amorphous simvastatin is characterized by its XRPD pattern substantially in accordance with FIG. 1.

The process for the preparation of novel amorphous simvastatin of the present invention is simple, eco-friendly and easily scaleable.

The invention is further described by reference to the following examples, which set forth in detail certain aspects and embodiments of the preparation of compounds and compositions of the present invention. It will be apparent to those skilled in the art, that many modifications, both to materials and methods, can be practiced without departing from the purpose and intent of this invention. The examples that follow are not intended to limit the scope of the invention as described hereinabove or as claimed below.

### EXAMPLE 1

Preparation of Amorphous Simvastatin by Spray Drying

5 g of simvastatin was charged into a round bottom flask equipped with a half-moon Teflon blade agitator at 30° C. To this 60 ml of methanol was charged at 30° C. The resulting mixture was stirred for about 15 minutes at 30° C. After dissolution of the solids, solution was filtered on a Büchner funnel through flux calcined diatomaceous earth. The resulting particle-free solution was dried in a spray drier under the following set of conditions: feed rate, 4.8 ml per minute; air inlet temperature 75-80° C., air outlet temperature 30-35° C., aspiration 5 cm water column, a nitrogen pressure of 2 kg/cm² to afford amorphous simvastatin, as was observed from the XRPD pattern of FIG. 1, where the vertical axis is intensity and the horizontal axis is the 2θ angle, in degrees.

### EXAMPLE 2

Preparation of Amorphous Simvastatin by Buchi Distillation

5.0 g of simvastatin was charged into a round bottom flask equipped with a half-moon Teflon blade agitator at 30° C. To this 60 ml of methanol was charged at 30° C. The resulting mixture was stirred for about 15 minutes at 30° C. After dissolution of the solids, solution was filtered on a Büchner funnel through flux calcined diatomaceous earth. The resulting particle free solution was distilled to dryness in a Buchi Rotovap flask under a reduced pressure of 650 mm Hg at 40-43° C. The product was dried for 30 minutes in the Buchi flask under reduced pressure at 40-41° C. to afford amorphous simvastatin, as was observed from the XRPD pattern.

### EXAMPLE 3

Preparation of Amorphous Simvastatin Composition with Povidone

4.0 g of simvastatin and 4.0 g of polyvinylpyrrolidone (povidone K 30) were charged into a round bottom flask equipped with a half-moon Teflon blade agitator at 30° C. To this 40 ml of methanol was charged at 30° C. The resulting mixture was stirred for about 15 minutes at 30° C. After dissolution of the solids, the solution was filtered on a Büchner funnel through flux calcined diatomaceous earth. The resulting particle-free solution was distilled to dryness in a Buchi Rotovap flask under reduced pressure at 40-43° C. The product was dried for 60 minutes in the Buchi flask under reduced pressure at 40-45° C. to afford a stable composition comprising amorphous simvastatin, as was observed from the XRPD pattern.

We claim:

1. Amorphous simvastatin.

2. A molecular dispersion of amorphous simvastatin in a pharmaceutically acceptable carrier.

3. The molecular dispersion of claim 2, wherein a pharmaceutically acceptable carrier comprises a polyvinylpyrrolidone.

4. A process for preparing amorphous simvastatin, comprising rapidly removing solvent from a solution of simvastatin.

5. The process of claim 4, wherein a solution of simvastatin comprises a pharmaceutically acceptable carrier.

6. The process of claim 4, wherein a solution of simvastatin comprises a polyvinylpyrrolidone.

7. The process of claim 4, wherein solvent is removed by spray drying.

8. The process of claim 4, wherein solvent is removed by rotational evaporation.

9. The process of claim 4, wherein solvent is removed by agitated thin film drying-vertical.

10. The process of claim 4, wherein solvent is removed by spin-flash drying.

11. The process of claim 4, wherein solvent is removed by fluid bed drying.

12. The process of claim 4, wherein solvent is removed by lyophilization.

13. The process of claim 4, wherein a solution of simvastatin comprises an alcohol.

14. The process of claim 4, wherein a solution of simvastatin comprises methanol.

15. The process of claim 4, wherein a solution of simvastatin comprises methanol and a polyvinylpyrrolidone.
